(19) United States
(12) Patent Application Publication
(10) Pub. No.: US 2008/0255767 A1
(43) Pub. Date: Oct. 16, 2008

(54) Method and Device For Detection of Splice Form and Alternative Splice Forms in Dna or Rna Sequences

(75) Inventors: Gunnar Ratsch, Tubingen (DE); Soren Sonnenburg, Berlin (DE); Klaus-Robert Muller, Berlin (DE); Bernhard Scholkopf, Tubingen (DE)

Correspondence Address: THE WEBB LAW FIRM, P.C., 700 KOPPERS BUILDING, 436 SEVENTH AVENUE, PITTSBURGH, PA, 15219, US

(73) Assignees: FRAUNHOFER-GESELLSCHAFT ZUR FORDERUNG DER ANGEWANDTEN, Munchen (DE); MAX-PLANCK GESELLSCHAFT ZUR FORDERUNG DER, WISSENSCHAFTEN .E.V., BERLIN, Munchen (DE)

(21) Appl. No.: 11/597,218

(22) PCT Filed: May 25, 2005
(86) PCT No.: PCT/EP2005/005783

§ 371 Date: Nov. 21, 2006

(30) Foreign Application Priority Data
May 26, 2004 (EP) 04012454.7
May 6, 2005 (EP) 05090129.7

(51) Int. Cl. G01N 33/48; G06F 19/00

(52) U.S. Cl. 702/20

(57) ABSTRACT

The invention relates to a method and a device for detection of splice sites in DNA or RNA sequences comprising three steps: a) examining a training set of sequences comprising DNA or RNA sequences with known splice sites by an automated, discriminative training device for detecting splicing patterns, especially in a predetermined window around the known splice sites; b) scanning a sequence comprising DNA or RNA sequences containing unknown splice sites for the occurrence of the splicing patterns detected in step a); and c) calculation of a cumulative splice score in dependence of a maximization of the margin between the true splice forms and all wrong splice forms in the sequence. The invention also relates to a method and a device for detection of splice forms and alternative splice forms in DNA or RNA sequences.

The invention relates to a method for detection of a splice form in DNA or RNA sequences according to claim 1 and a method for detection of splice forms and alternative splice forms in DNA or RNA sequences according to Claims 2 and 7. The invention also relates to a device for detection of a splice form in DNA or RNA sequences according to claim 20 and a device for detection of splice forms and alternative splice forms in DNA or RNA sequences according to Claims 21 and 22.

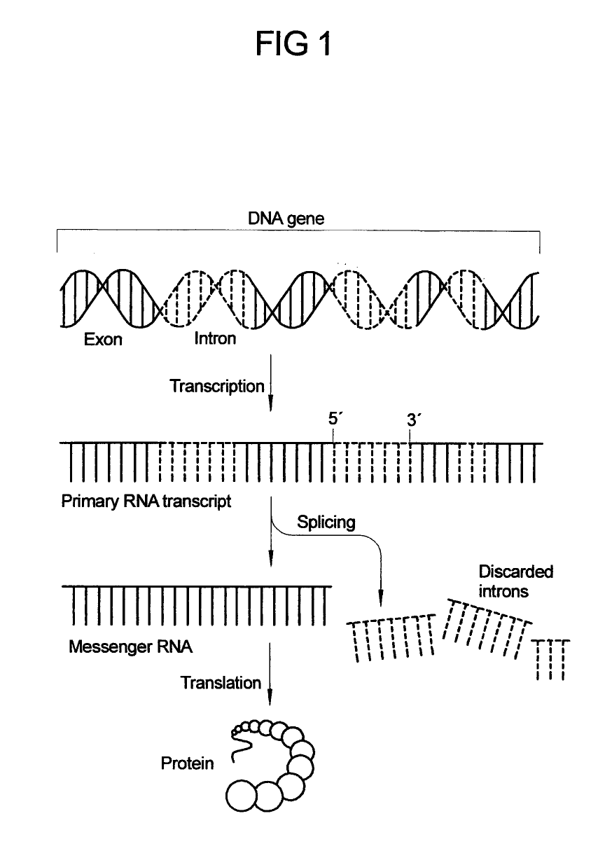

Eukaryotic genes contain intervening usually non-coding sequences in the genomic DNA designated as introns. Those introns are excised from a gene transcript with the concomitant ligation of the flanking segments called exons during a process known as splicing (FIG. 1, Scientific American, April 2005, pp. 42).

For example, the genome of the soil nematode C. elegans contains around 100 million base pairs with 22,259 estimated genes when the alternatively spliced forms are included. Only 4,878 (21.9%) genes have been confirmed by cDNA and EST sequences. Of the remaining gene models, primarily based on computational predictions, 11,857 (53.3%) have been partially confirmed and 5,524 (24.8%) lack any transcriptional evidence.

Methods for predicting splice sites and hence genes are known. Those known methods are based on alignment or probabilistic learning systems, which typically rely on homology and evolutionary information using reading frame information, exon counts, repeat masking, similarity to known genes and proteins, or any other evolutionary information (Ref 23 to 30 in Appendix A). These systems, however, do not give an accurate annotation of splice sites and hence genes.

However, an accurate prediction of splice sites is desirable, for application in medicine, drug discovery and molecular biology.

An object of the invention is therefore to provide a method which enables a person skilled in the art to accurately predict splicing sites in genomic DNA or unspliced RNA sequences.

This object can be achieved by providing a method according to Claim 1 and a device according to Claim 20.

The method according to Claim 1 for the detection of splice sites in a genomic DNA or RNA comprises three steps:

a) Examining a training set of sequences comprising DNA or RNA sequences with known splice sites by an automated, discriminative training device for detecting splicing patterns, especially in a predetermined window around the known splice sites;

b) Scanning a sequence comprising DNA or RNA sequences containing unknown splice sites for the occurrence of the splicing patterns detected in step a); and

c) Calculation of a splice score in dependence of a maximisation of the margin between the true splice forms and all wrong splice forms in the sequence, whereby true splice forms refer to known splice forms and wrong splice forms refer to variations of known splice forms. The calculation is carried out by using a large margin algorithm.

The derivation of the training set is described in detail e.g. in Appendix B, Section 1. One important feature of a good training set is relatively low noise-level.

The computation of the cumulative splice score and the definition of splice forms are e.g. described in Appendix B, Section 2.3.

The goal is to discover the unknown formal mapping from genomic DNA or unspliced pre-mRNA to mature mRNA given a sufficient number of examples for “training”.

This is achieved in the present invention by employing machine learning techniques, especially by employing a Support Vector Machine (SVM) to model and predict how the splicing process acts and to obtain at least one training set of sequences.

Furthermore, a device for the detection of at least one splice site in a DNA or RNA sequence according to Claim 20 is part of the present invention. The device comprises:

a) An automated, discriminative training device for detecting splicing patterns, especially in a predetermined window around the known splice sites, in a training set of sequences comprising EST, RNA sequence and/or cDNA with known splice sites;

b) A scanning device for scanning a second sequence comprising premature RNA (unspliced mRNA) containing unknown splice sites for the occurrence of the splicing patterns detected in step a); and

c) A calculation device for automatically calculating a cumulative splice score in dependence of a maximisation of the margin between the true splice forms and all wrong splice forms.

The device can be implemented as software running on a computing device and/or as hardware, e.g. a computer chip.

Unlike the known generative methods, a.k.a. probabilistic methods, the present invention does not require the calculation of continuous probability densities and is not based on the maximization of some probabilistic likelihood function. The calculation is much simplified by the introduction of discriminative.

In a preferred embodiment of the invention support vector machine (SVM) classifiers are used for detecting the starts and ends of introns, as well as for recognizing the exon and intron content. This classification is learned from sequences with known splice sites.

SVMs have their mathematical foundations in a statistical theory of learning and attempt to discriminate two classes by separating them with a large margin (margin maximization).

They employ similarity measures referred to as kernels which are designed for the classification task. It is desirable that the kernels compare pairs of sequences in terms of their matching substring motifs.

It is also preferable that SVMs are trained by solving an optimization problem involving labeled training examples—true splice sites (positive) and decoys (negative).

SVMs can be used to classify sequences into two classes, e.g. constitutive splice sites vs. non-splice sites. In a first step one obtains a training set of true and false sites by extracting one or several windows of the considered sequences around the splice sites. By using the SVM learning machine in the next step a SVM classifier is obtained that is able to classify yet unclassified sites, e.g. of another sequence, into true and false sites.

It is further desirable, that the SVM splice detectors are scanned over DNA or RNA sequences, and, in a second step, their predictions are combined to form the overall splicing prediction. It is implemented using a state based system similar to Hidden-Markov model based gene finding approaches (see also References 15-20 in Appendices A & B).

An advantage of the method and device according to the invention is described as follows. The learning algorithm determines the parameters of a splice score function that is able to score splice forms for a given sequence. Unlike previous learning systems that usually maximize some probabilistic likelihood function, the algorithm is based on the comparison of known true, i.e. known or putative, splice sites or splice forms with deviating, i.e. wrong, splice sites or splice forms. The system has the goal to find the parameters of the splice score function such that the score difference between the score of the true splice form and any other splice form is simultaneously as large as possible for all training sequences. This approach turns out to overcome many problems of the Hidden-Markov models commonly used for gene finding.

One preferred embodiment (method and device) is described in Appendix A.

Another advantage of the invention is that information might be used which is in principle available to the cellular splicing machinery, such as sequence-based splice site identification via the splicing factors U1-U6, lengths of exons and introns via physical properties of mRNA, and intron as well as exon sequence content i.e. via splice enhancers.

The invention does not necessarily utilize reading frame information, exon counts, repeat masking, similarity to known genes and proteins, or any other evolutionary information.

The invention according to Claim 1 and Claim 20 is described in Appendix A giving an example of splice site detection mainly in C. elegans unspliced mRNAs. Appendix B describes the algorithmic mechanism employed in the detection of the splice sites.

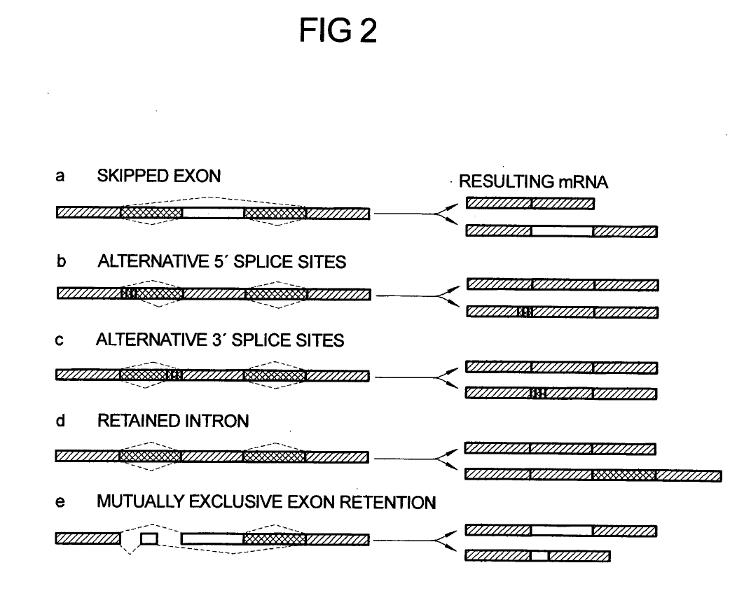

The primary sequence of an eukaryotic gene containing exons as coding sequences and introns as non-coding sequences can not only be edited in one way, but in several, alternative ways (see FIG. 2, Scientific American, April 2005, pp. 42).

Alternative splicing is a process through which one gene can generate several distinct mRNAs and proteins. It can be specific to a tissue, developmental stage or a condition such stress.

Traditional methods for computational recognition of alternative splicing are solely based on expressed sequences (see Ref. 7, Appendix C) or conservation patterns to another organism (see Ref. 22, Appendix C) have been taken into account. However, this is only possible for a fraction of exons, e.g. in human, as exons are frequently not conserved.

It is therefore also an object of the present invention to provide a method and a device that accurately distinguishes constitutively from alternatively spliced exons and use only information that might also be used by the cellular splicing machine including features derived from the exon and intron lengths and features based on the pre-mRNA sequence.

This object can be achieved by employing a method according to Claims 2 and 7 and a device according to Claims 21 and 22.

The method for the identification of one splice form and/or alternative splice forms each comprising predictions of exon locations in DNA or RNA sequences according to Claim 2 comprises:

a) a training set of DNA or RNA sequences with putative splice sites e.g. derived from corresponding EST and/or cDNA sequences (see also U.S. Pat. No. 6,625,545) or a curated genome annotation (see ENCODE project under http://www.genome/gov) is examined by an automated, preferably discriminative training device for detecting splicing patterns, especially using predetermined windows around the putative splice sites, whereby the splicing pattern may include information of alternative splice events e.g. exon skipping or intron retention, alternative exon start or end usage or the existence of regulative elements;

b) a second training set of DNA or RNA sequences with putative splice forms, whereby the training sets of a) and b) can be the same, is examined by an automated, discriminative training device using splice patterns detected in step a) leading to a calculation device to automatically assign scores to a splice form and/or a group of alternative splice forms preferably in dependence of the maximization of the margin between the putative splice forms (or groups of them) and putatively wrong splice forms or groups of splice forms of sequences in the training set applying a Large Margin based Learning Algorithm;

c) a sequence comprising RNA or DNA with unknown and/or putative splice sites is scanned for the occurrence of the splicing patterns detected in step a); and

d) using the device that assigns scores in dependence of the result of step c), a splice form or group of alternative splice forms is predicted in dependence of the said scores, comprising a set of splice forms associated with a RNA or DNA sequence, especially when used to identify several alternative or only one mRNAs and/or proteins associated with a RNA or DNA sequence.

A group of splice forms as used in b) can be for instance the set of splice forms which are the result of alternative splicing (for instance generated by alternative exon or intron usage and/or alternative starts or ends of exons).

The invention preferably employs two algorithms for the identification of alternatively spliced exons based on confirmed exons and introns. The first algorithm uses an appropriately designed Support Vector Kernel as a SVM that is able to deal with DNA sequences in order to learn about the sequence features near the 3′ and 5′ end of alternatively spliced exons. The aim is to classify known exons into alternatively and constitutively spliced exons.

However, if this first algorithm is applied for instance to EST confirmed regions, the exon might be skipped in the existing sequencing results and hence is not found.

Therefore a second algorithm is introduced that not only specifies an alternatively spliced exon, but it also enables the detection of its accurate location within an intron. This algorithm can be applied to scan over all EST confirmed introns for skipped exons.

A preferred embodiment of the invention is described in Appendix C.

The method detects alternatively spliced exons by applying a classifier based on SVM's classifying exons in constitutively or alternatively spliced forms, i.e. if exons might be skipped. This requires a known splice form, i.e. the exon has to be known beforehand.

The goal of this method is to find splice forms and alternatively spliced exons simultaneously.

In the simplest case only alternatively splice forms differing from each other by skipped exons would be detected. A group of splice forms can be a list of skipped exons with additional information regarding which exons might be skipped, whereby defining a number of potential splice forms and hence transcripts.

In a more general case also information regarding intron retention as well as alternative starts and ends would be added. For this purpose, additional classifiers recognizing such splice sites are required. A group of splice forms would be than available by the listed exons and introns, whereby possibly skipped exons and possibly retained introns, exon starts with alternative start sites as well as exon ends with alternative end sites are marked. Ideally, a group of splice forms also contains information, how the different alternative splice events collude as for instance in case of exclusively used exons.

A scoring function is calculated by applying a Large Margin Learning Algorithm based on the detectors for the different alternative splice events. It determines the parameters of the scoring function—simultaneously for all training examples—such that the margin, i.e. difference, between the scores of a true group of splice forms and any deviating splice form group is maximized.

In a preferred embodiment steps a) & b) and/or c) & d) are integrated into one combined step.

Furthermore, partial information about the sequences of the training set is used, especially in order to improve the prediction accuracy and when used repetitively in order to complete missing information about the training sequences.

A combination with putative transcription starts, especially promoters or trans-splice sites, and transcription ends, especially a polyA signal, is employed to infer sets of mRNA sequences and/or proteins associated with one or several locations on the RNA or DNA sequence.

This includes but is not limited to the information about existing annotations of RNA or DNA sequences comprising putative transcript starts and ends. This information is used in order to identify sets of mRNA sequences and/or proteins from the RNA and/or DNA sequence.

The method for the detection of alternative splice forms is described in Appendix C.

The device for the detection of at least one splice form in a DNA or RNA sequence according to Claim 21 comprises:

a) an automated, preferably discriminative training device for detecting splicing patterns, especially in a predetermined window around putative splice sites, in a training set comprising RNA or DNA sequences with putative splice sites, whereby the splicing patterns may include information about alternative splice events, e.g. for instance exon or intron skipping, alternative exon start or end usage;

b) a discriminative training device leading to a calculation device that automatically assigns scores to a splice form and/or a group of splice forms preferably in dependence of the maximization of the margin between putative splice forms (or groups of them) and putatively wrong splice forms associated with sequences in a second training set of DNA or RNA sequences with putative splice forms;

c) a scanning device for scanning a RNA and/or DNA sequence containing unknown and/or putative splice sites for the occurrence of the splicing patterns detected by the device in step a).

d) a calculation device for automatically calculating a score (as generated by device in step b) to splice forms and/or groups of splice forms in a RNA and/or DNA sequence in dependence of device in step c), especially for using it to identify a set of splice forms (and hence mRNAs and/or proteins) associated to a RNA or DNA sequence.

The device for the detection of alternative splice forms is described in Appendix C.

Further advantages and features of the methods and devices according to the invention are pointed out by the following figures and examples.

FIG. 1 showing a the principle of splicing;

FIG. 2 showing the principle of alternative splicing;

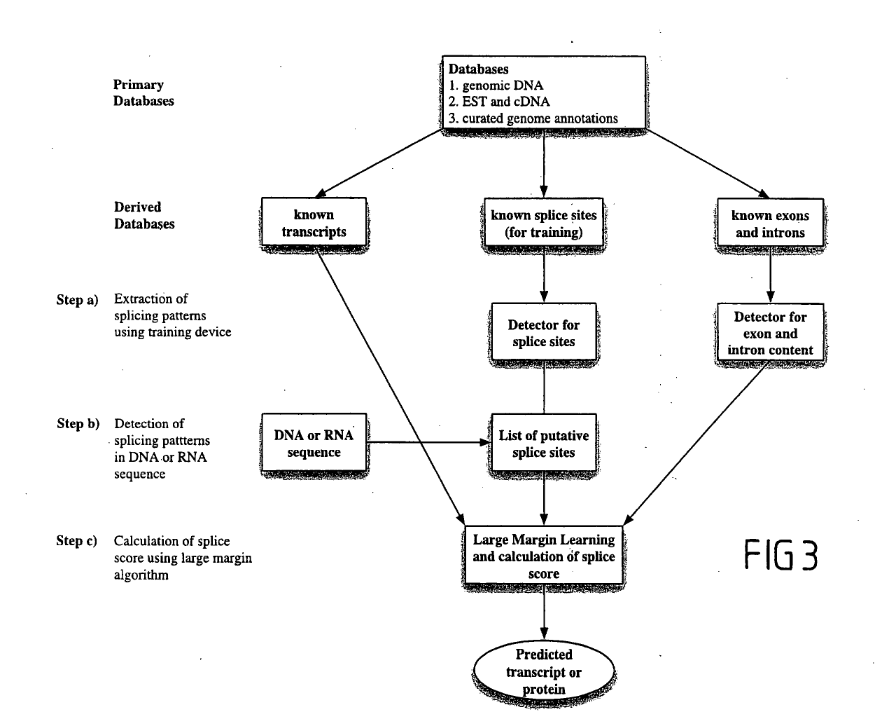

FIG. 3 showing the basic scheme of a first embodiment of the invention;

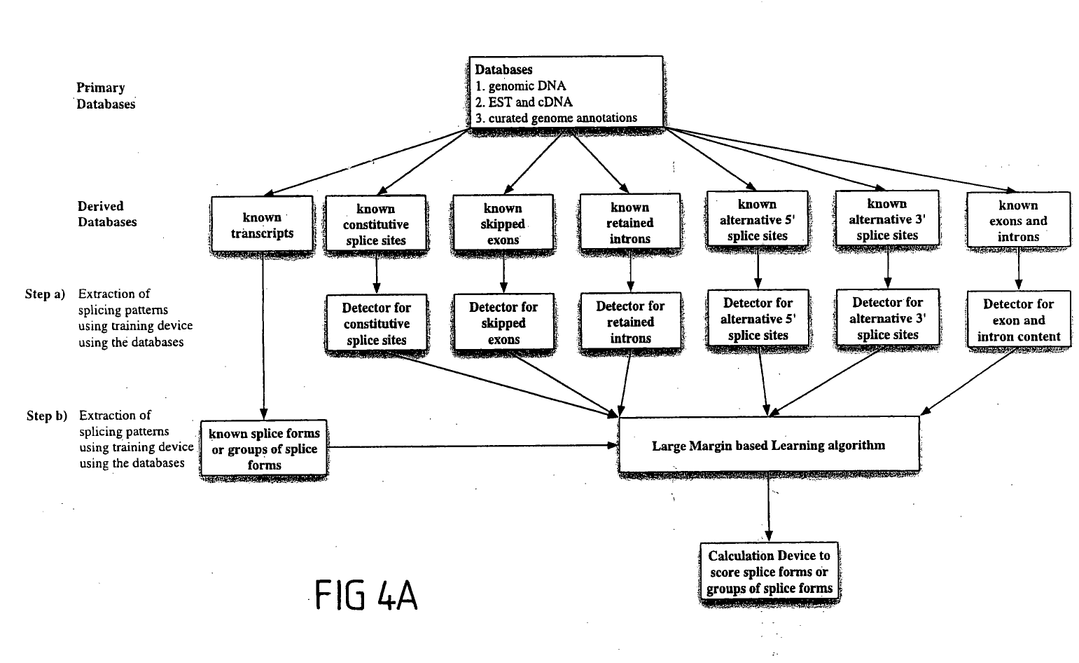

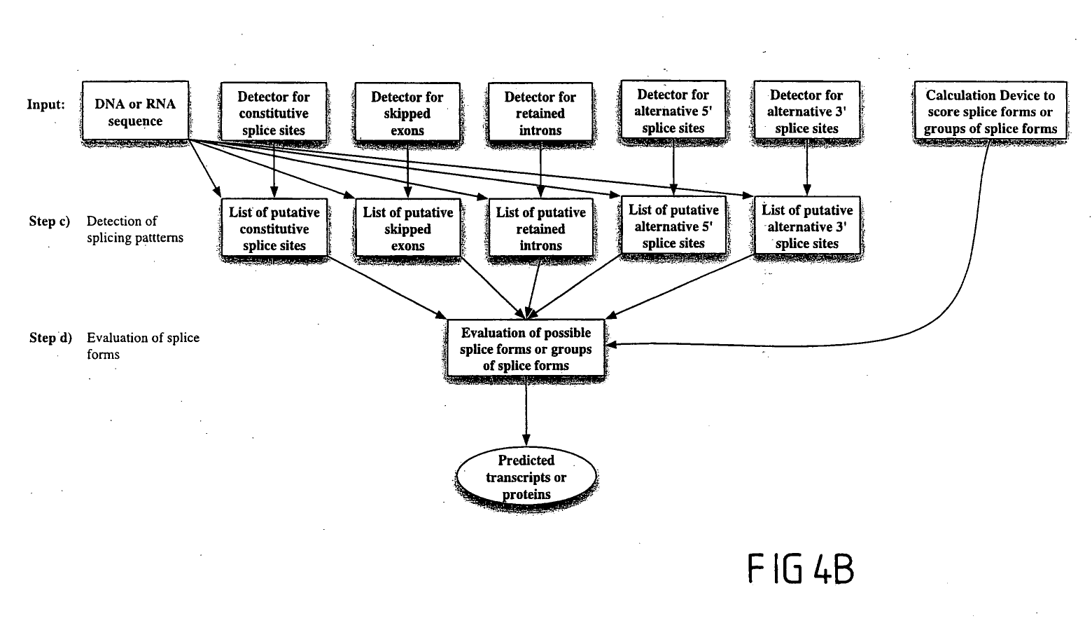

FIG. 4A,B showing the basic scheme of the second embodiment of the invention;

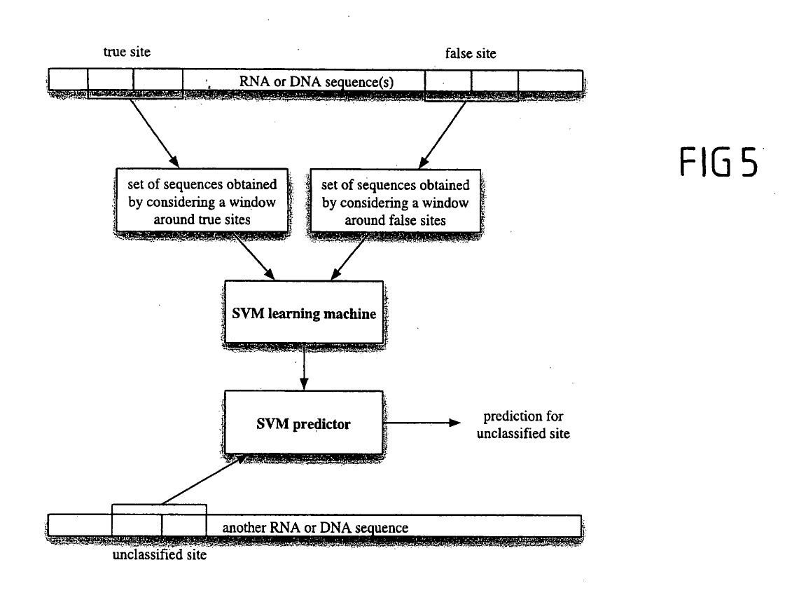

FIG. 5 showing the basic scheme the inclusion of an SVM mechanism in a further embodiment.

FIG. 1 shows the classical view of eukaryotic gene expression. A DNA sequence is transcribed into a single-stranded RNA copy. The primary RNA transcript is then spliced by the cellular machinery, whereby introns are removed. Each intron is distinguished by its 5′ end and 3′ end splice sites. The remaining exons are ligated to one mRNA version of the gene that will be translated into a protein by the cell.

FIG. 2 describes the alternative splicing approach. A primary transcript of a eukaryotic gene can be edited in several different ways. The different splicing activities are indicated in FIG. 2 by dashed lines. The splicing events can proceed as in a) where an exon is left out, as in b) where an alternative 5′ splice site is detected or in c) where an alternative 3′ splice site is detected by the splicing machinery. Furthermore, an intron may be retained in the final mRNA transcript as in d) or exons may be retained on a mutually exclusive basis.

FIG. 3 shows a flow scheme comprising a first embodiment of the invention. In a first step a) known splice sites, exons and introns are extracted from data bases. A SVM classifier is then trained for the two kinds of splice sites, i.e. exon start and end, whereby the classifier is able to detect these splice sites. Moreover, the content of exon(s) and intron(s) is analysed by SVMs in order to detect patterns in exon(s) or intron(s). In the next step b) a second training set, specifically of non-alternative spliced transcripts, is used in order to define splice forms. These splice forms are then analyzed in step c) by applying the Large Margin Algorithm from which a scoring function for splice forms is derived.

The parameters of the splice score function are adjusted in such a way that the margin is maximized, i.e. the difference between the functional value for the correct, known splice form and the wrong, deviating splice form is maximized. In step b) the subjected sequence is analyzed and a list of potential splice sites is created. Any, from such a list emerging splice form is evaluated by the splice score function. Typically, the maximum value is selected providing the basis for predicting the splice form of the given sequence. In the last step, the sequence of the spliced mRNA and, where appropriate, protein might be deduced from the predicted splice form.

FIGS. 4a) and 4b) provide a flow scheme comprising a second embodiment of the invention. In a first step a) known splice sites and information about known alternative splice events, e.g. skipped exons, retained introns, alternative 5′ and 3′ splice sites, are extracted from data bases. A SVM classifier is trained for every possible event in this step. In the following step b) a second training set of possibly alternative transcripts is used to define splice forms or groups of splice forms, which are then analyzed by the Large Margin Algorithm from which a score function is derived. The parameters are again adjusted in such a way that the margin is maximized, i.e. the difference between the functional value for the correct, known splice form and the wrong, deviating splice form is maximized.

In steps c) and d) a sequence is subjected to analysis. Lists of potential splice sites or other alternative splice events are created. Any, from such a list emerging splice form is evaluated by the splice score function. Typically, the maximum value is selected providing the basis for predicting the splice form of the given sequence. In the last step, the sequence of the spliced mRNA and, where appropriate, protein might be deduced from the predicted splice form.

In FIG. 5 a scheme is shown which depicts the generation of a SVM classifier using a SVM learning machine. SVMs are used to classify sequences in two classes. The two classes might comprise constitutive splice sites vs. non-splice sites, alternatively spliced or skipped exons vs. constitutively spliced exons, alternative exon starts vs. constitutive exon starts and others. In a first step a training set of true and false sites, i.e. examples and counter examples, are obtained by extracting one or several windows of the considered sequences around the splice sites, whereby true and false sites in the sequence must be known for training. Using the SVM learning machine a SVM classifier is obtained that is able to classify so far unclassified sites, e.g. of another sequence, into true and false sites.

1-33. (canceled)

34. A method for the detection of a splice form in a DNA or RNA sequences, comprising:
a) examining a training set of sequences comprising DNA or RNA sequences with known splice sites by an automated, discriminative training device for detecting splicing patterns in a predetermined window around the known splice sites;
b) scanning a sequence comprising DNA or RNA sequences containing unknown splice sites for the occurrence of the splicing patterns detected in step a); and
c) calculating automatically a splice score in dependence of a maximization of the margin between the scores of true splice forms and all wrong splice forms in the sequence, wherein true splice forms refer to known splice forms and wrong splice forms refer to variations of known splice forms.

35. A method for the identification of one splice form and/or several alternative splice forms each comprising predictions of exon locations in DNA or RNA sequences, comprising:
a) examining a training set of DNA or RNA sequences with putative splice sites by an automated, discriminative training device for detecting splicing patterns using predetermined windows around the putative splice sites, wherein the splicing patterns can include information of alternative splice events, such as exon skipping or intron retention, alternative exon start or end usage or existence of regulative elements;
b) examining a second training set of DNA or RNA sequences with putative splice forms by an automated, discriminative training device using splice patterns detected in step a), leading to a calculation device to automatically assign scores to a splice form and/or a group of alternative splice forms in dependence of the maximization of the margin between the putative splice forms or groups of them and putatively wrong splice forms of sequences or groups of them in the training set, wherein a Large Margin based Learning algorithm is applied;
c) scanning a sequence comprising RNA or DNA with unknown and/or putative splice sites for the occurrence of the splicing patterns detected in step a); and
d) predicting a splice form or group of alternative splice forms, using the device that assigns scores in dependence of the result of step c), in dependence of the said scores by maximizing or minimizing a function of the scores, comprising a set of splice forms associated with a RNA or DNA sequence when used to identify several alternative or only one mRNAs and/or proteins associates with a RNA or DNA sequence.

36. The method according to claim 35, whereby steps a) and b) and/or c) and d) are integrated into one combined step.

37. The method according to claim 35, wherein partial information about the sequences of the training set is used in order to improve the prediction accuracy, and is used repetitively in order to complete missing information about the training sequences.

38. The method according to claim 35, wherein a combination with putative transcription starts, especially promoters or trans-splice sites, and ends, especially a polyA signal, is used to infer sets of mRNA sequences and/or proteins associated with one or several locations on the RNA or DNA sequence.

39. The method according to claim 38, wherein information about existing annotations of a RNA or DNA sequence comprising putative transcript starts and ends is used in order to identify sets of mRNA sequences and/or proteins from the RNA and/or DNA sequence.

40. A method for the detection of at least one splice form and/or at least one alternative splice form in RNA and DNA sequences, each comprising predictions of exon locations in DNA or RNA sequences, comprising:
a) examining a first training set of DNA or RNA sequences with putative splice sites by an automated training device for detecting splicing patterns;
b) examining a second training set of DNA or RNA sequences with putative splice forms by an automated, discriminative training device using splice patterns detected in step a), leading to an automatic assignment of scores to at least one splice form and/or a group of alternative splice forms by a calculation device;
c) scanning a sequence comprising RNA or DNA with unknown and/or putative splice sites for the occurrence of the splicing pattern(s) detected in step a); and
d) calculating at least one splice form and/or at least one alternative splice form in dependence of the step b) assigned scores by using the calculation device and in dependence of the results obtained in step c), wherein at least one set of splice forms associated with a RNA or DNA sequence is provided.

41. The method according to claim 40, wherein an automated discriminative training device is used for detecting splice patterns in step a).

42. The method according to claim 40, wherein the splice patterns are detected in step a) by using a predetermined window around the putative splice sites.

43. The method according to claim 40, wherein the splicing patterns detected in step a) comprise sequence patterns, alternative start and end of exon(s), skipping of exon(s) and retaining of intron(s) and/or existence of regulative element(s).

44. The method according to claim 40, wherein the DNA or RNA sequences with putative splice forms are examined in step b) in dependence of the maximization of the margin between the putative splice forms or groups of splice forms and putative wrong splice forms of sequences in the training set.

45. The method according to claim 40, wherein at least one splice form and/or at least one alternative splice form is calculated in step d) by maximizing or minimizing a function of the step c) assigned scores.

46. The method according to claim 40, wherein in step d) at least one mRNA, several alternatively spliced mRNA's and/or proteins associated with a splice RNA and/or DNA sequence are provided.

47. The method according to claim 40, wherein steps a) and b) and/or c) and d) are integrated into one combined step.

48. The method according to claim 40, wherein the training set(s) comprise partial sequence information in order to improve the prediction accuracy.

49. The method according to claim 40, further comprising providing missing information of the training set(s) by an iterating application.

50. The method according to claim 40, wherein information of putative transcriptional starts such as promoters and/or trans-splice sites, and transcriptional ends such as polyA-signals, is used to infer sets of mRNA sequences and/or proteins associated with one or several locations on the RNA or DNA sequence.

51. The method according to claim 50, wherein information of existing annotations or RNA or DNA sequences comprising transcriptional starts and ends is used.

52. The method according to claim 40, wherein at least one training set is analyzed with a Support Vector Machine.

53. A device for the detection of at least one splice site in a DNA or RNA, comprising:
a) an automated, discriminative training device for detecting splicing patterns in a predetermined window around the known splice sites, in a training set of sequences comprising EST, RNA sequence and/or DNA with known splice sites;
b) a scanning device for scanning another sequence comprising DNA or RNA sequences containing unknown splice sites for the occurrence of the splicing patterns detected in step a); and
c) a calculation device for automatically calculating a splice score in dependence of a maximization of the margin between the true splice forms and all wrong splice forms.

54. A device for the detection of at least one splice form in a DNA or RNA sequence, comprising:
a) an automated, discriminative training device for detecting splicing patterns in a predetermined window around putative splice sites in a training set comprising RNA or DNA sequences with putative splice sites, wherein splicing patterns can include information about alternative splice events such as exon skipping or intron retention, alternative exon start or end usage;
b) a discriminative training device leading to a calculation device that automatically assigns scores to a splice form and/or a group of splice forms in dependence of the maximization of the margin between putative splice forms or groups of them and putatively wrong splice forms associated with sequences in a second training set of DNA or RNA sequences with putative splice forms;
c) a scanning device for scanning a RNA and/or DNA sequence containing unknown and/or putative splice sites for the occurrence of the splicing patterns detected by the device in step a); and
d) a calculation device for automatically calculating a score generated by the device in step b) to splice forms and/or groups of splice forms in a RNA and/or DNA sequence in dependence of the device in step c), wherein it is used to identify a set of splice forms such as mRNAs and/or proteins associated to a RNA or DNA sequence.

55. A device for the detection of at least one splice form in a DNA or RNA sequence, comprising:
a) an automated training device for detecting splicing patterns in a training set comprising RNA or DNA sequences with putative splice sites;
b) a discriminative training device leading to a calculation device automatically assigning scores to at least one splice form and/or a group of splice forms and putatively wrong splice forms associated with sequences in a second training set of RNA or DNA sequences with putative splice forms;
c) a scanning device for scanning a RNA and/or DNA sequence containing unknown and/or putative splice sites for the occurrence of the splicing pattern(s) detected in step a); and
d) a calculation device for automatically calculating a score generated by the device in step b) of at least one splice form and/or groups of splice forms in a RNA or DNA sequence in dependence on the device in c).

56. The device according to claim 55, wherein an automated discriminative training device is used for detecting splice patterns in step a).

57. the device according to claim 55, wherein the splice patterns are detected in step a) by using a predetermined window around the putative splice sites.

58. The device according to claim 55, wherein the splicing patterns detected in step a) comprise sequence patterns, alternative starts or ends of exon(s), skipping of exon(s), retention of intron(s) and/or existence of regulative element(s).

59. The device according to claim 55, wherein the DNA or RNA sequences with putative splice forms are examined in step b) in dependence of the maximization of the margin between the putative splice forms or groups of splice forms and putative wrong splice forms of sequences in the training set.

60. The device according to claim 55, wherein in step d) at least one mRNA, several alternatively splice mRNAs, a set of splice forms and/or proteins associated with a splice RNA and/or DNA sequence are provided.

61. The device according to claim 55, wherein steps a) and b) and/or c) and d) are integrated into one combined step.

62. The device according to claim 55, wherein the training set(s) comprise partial sequence information in order to improve the prediction accuracy.

63. The device according to claim 55, wherein an iterating application of the device provides missing information of the training set(s).

64. The device according to claim 55, wherein information of putative transcriptional starts, promoters and/or trans-splice sites, and transcriptional ends such as polyA-signals, is used for the device to infer sets of mRNA sequences and/or proteins associated with one or several locations on the RNA or DNA sequence.

65. The device according to claim 64, wherein information of existing annotations or RNA or DNA sequences comprising transcriptional starts and ends is used for the device.

66. The device according to claim 55, wherein the training device comprises a support vector machine.


SEQUENCE LISTING (166 sequences)

SEQ ID NO: 1 - DNA, 21 bp, Artificial Sequence
  Synthetic construct
ttttatcgcagattgtcatcg

SEQ ID NO: 2 - DNA, 21 bp, Artificial Sequence
  Synthetic construct
ggatttggttttctggatgct

SEQ ID NO: 3 - DNA, 23 bp, Artificial Sequence
  Synthetic construct
cagattgtcatcgaactttatcg

SEQ ID NO: 4 - DNA, 18 bp, Artificial Sequence
  Synthetic construct
tatcgtctccgggctcag

SEQ ID NO: 5 - DNA, 19 bp, Artificial Sequence
  Synthetic construct
catcactcattccagcccc

SEQ ID NO: 6 - DNA, 18 bp, Artificial Sequence
  Synthetic construct
cgtttcgcggagaactgt

SEQ ID NO: 7 - DNA, 21 bp, Artificial Sequence
  Synthetic construct
cattccagcccctcatactct

SEQ ID NO: 8 - DNA, 21 bp, Artificial Sequence
  Synthetic construct
tgtcgacggagtttgatctac

SEQ ID NO: 9 - DNA, 21 bp, Artificial Sequence
  Synthetic construct
tgttgtcagttcttgctttcc

SEQ ID NO: 10 - DNA, 19 bp, Artificial Sequence
  Synthetic construct
tccgcatacatacccagtg

SEQ ID NO: 11 - DNA, 22 bp, Artificial Sequence
  Synthetic construct
ttcttgctttcctactcagcaa

SEQ ID NO: 12 - DNA, 18 bp, Artificial Sequence
  Synthetic construct
cagtgggatcagctcgga

SEQ ID NO: 13 - DNA, 21 bp, Artificial Sequence
  Synthetic construct
gagcacagtaaacttggtggc

SEQ ID NO: 14 - DNA, 19 bp, Artificial Sequence
  Synthetic construct
gattgaacgggagccatgt

SEQ ID NO: 15 - DNA, 21 bp, Artificial Sequence
  Synthetic construct
gtaggctccgttgctatcgtt

SEQ ID NO: 16 - DNA, 20 bp, Artificial Sequence
  Synthetic construct
agccatgtgggaaattggat

SEQ ID NO: 17 - DNA, 21 bp, Artificial Sequence
  Synthetic construct
gctttctcgccatgtattgtc

SEQ ID NO: 18 - DNA, 19 bp, Artificial Sequence
  Synthetic construct
atctaccggtggcatttcc

SEQ ID NO: 19 - DNA, 21 bp, Artificial Sequence
  Synthetic construct
attgtctatggtggttcggtg

SEQ ID NO: 20 - DNA, 21 bp, Artificial Sequence
  Synthetic construct
ttccaattgggatttgtcatc

SEQ ID NO: 21 - DNA, 21 bp, Artificial Sequence
  Synthetic construct
ttccaccaaacagtccagaac

SEQ ID NO: 22 - DNA, 21 bp, Artificial Sequence
  Synthetic construct
tgttacggtcgatgtctccat

SEQ ID NO: 23 - DNA, 21 bp, Artificial Sequence
  Synthetic construct
gaacaaattgtccttgggttg

SEQ ID NO: 24 - DNA, 21 bp, Artificial Sequence
  Synthetic construct
cattgcaggtgttgtcatcat

SEQ ID NO: 25 - DNA, 21 bp, Artificial Sequence
  Synthetic construct
ctttccatttttgcacatgac

SEQ ID NO: 26 - DNA, 21 bp, Artificial Sequence
  Synthetic construct
tgacgatattccagttgagca

SEQ ID NO: 27 - DNA, 23 bp, Artificial Sequence
  Synthetic construct
ttttgcacatgacaaagtatcgt

SEQ ID NO: 28 - DNA, 21 bp, Artificial Sequence
  Synthetic construct
tgagcactcgaaactgttgga

SEQ ID NO: 29 - DNA, 21 bp, Artificial Sequence
  Synthetic construct
tatggagattcacccgacgca

SEQ ID NO: 30 - DNA, 21 bp, Artificial Sequence
  Synthetic construct
gaaatcaaagcataacgcagc

SEQ ID NO: 31 - DNA, 23 bp, Artificial Sequence
  Synthetic construct
caaaggagttgtatattttccga

SEQ ID NO: 32 - DNA, 19 bp, Artificial Sequence
  Synthetic construct
gcagctagccaaacgacac

SEQ ID NO: 33 - DNA, 21 bp, Artificial Sequence
  Synthetic construct
tgaagggagaggaagcaattt

SEQ ID NO: 34 - DNA, 21 bp, Artificial Sequence
  Synthetic construct
cctgattggcaattctccata

SEQ ID NO: 35 - DNA, 23 bp, Artificial Sequence
  Synthetic construct
tttcaattgtgttcagtttttca

SEQ ID NO: 36 - DNA, 21 bp, Artificial Sequence
  Synthetic construct
ggtacagttggtttcggcata

SEQ ID NO: 37 - DNA, 20 bp, Artificial Sequence
  Synthetic construct
tgccatgtacattcagcacc

SEQ ID NO: 38 - DNA, 21 bp, Artificial Sequence
  Synthetic construct
gagagcgttccaaaatgattg

SEQ ID NO: 39 - DNA, 21 bp, Artificial Sequence
  Synthetic construct
acattcagcaccgatatgagc

SEQ ID NO: 40 - DNA, 23 bp, Artificial Sequence
  Synthetic construct
tggaaatactgataaggagcaca

SEQ ID NO: 41 - DNA, 21 bp, Artificial Sequence
  Synthetic construct
ctttcatgaacacccttgtca

SEQ ID NO: 42 - DNA, 22 bp, Artificial Sequence
  Synthetic construct
ttgtttccctcattttgacagt

SEQ ID NO: 43 - DNA, 21 bp, Artificial Sequence
  Synthetic construct
acccttgtcaatgaaatgctg

SEQ ID NO: 44 - DNA, 23 bp, Artificial Sequence
  Synthetic construct
tttgttttcacactcctgattga

SEQ ID NO: 45 - DNA, 20 bp, Artificial Sequence
  Synthetic construct
caatggactagccgatttcc

SEQ ID NO: 46 - DNA, 21 bp, Artificial Sequence
  Synthetic construct
gaatcacaacaacagaaccgc

SEQ ID NO: 47 - DNA, 21 bp, Artificial Sequence
  Synthetic construct
tccggaatgatgatgaatttg

SEQ ID NO: 48 - DNA, 21 bp, Artificial Sequence
  Synthetic construct
cagaaccgcaaagagagaagt

SEQ ID NO: 49 - DNA, 21 bp, Artificial Sequence
  Synthetic construct
ttttggaggtggaaatcatgt

SEQ ID NO: 50 - DNA, 23 bp, Artificial Sequence
  Synthetic construct
grgttgtattgccccatgttgtt

SEQ ID NO: 51 - DNA, 21 bp, Artificial Sequence
  Synthetic construct
tggaaatcatgttggaggagt

SEQ ID NO: 52 - DNA, 22 bp, Artificial Sequence
  Synthetic construct
tgttgtgtagacggtttcatca

SEQ ID NO: 53 - DNA, 21 bp, Artificial Sequence
  Synthetic construct
tacattgatgattggcgtcac

SEQ ID NO: 54 - DNA, 20 bp, Artificial Sequence
  Synthetic construct
aagcgattaaatcacgaccg

SEQ ID NO: 55 - DNA, 21 bp, Artificial Sequence
  Synthetic construct
tcacgacgaacattgtttcaa

SEQ ID NO: 56 - DNA, 21 bp, Artificial Sequence
  Synthetic construct
accggtggttgataaaccaga

SEQ ID NO: 57 - DNA, 18 bp, Artificial Sequence
  Synthetic construct
ggcgtggaaattgtggaa

SEQ ID NO: 58 - DNA, 21 bp, Artificial Sequence
  Synthetic construct
tgttggaggataggattgaca

SEQ ID NO: 59 - DNA, 20 bp, Artificial Sequence
  Synthetic construct
aaattgtggaaaacgcgaat

SEQ ID NO: 60 - DNA, 21 bp, Artificial Sequence
  Synthetic construct
tgacaattgtgcttccagtga

SEQ ID NO: 61 - DNA, 22 bp, Artificial Sequence
  Synthetic construct
ggacaccactagttcttcgacc

SEQ ID NO: 62 - DNA, 21 bp, Artificial Sequence
  Synthetic construct
gtcttcctatttgctccgcac

SEQ ID NO: 63 - DNA, 21 bp, Artificial Sequence
  Synthetic construct
cttcgaccactgaagttcctg

SEQ ID NO: 64 - DNA, 20 bp, Artificial Sequence
  Synthetic construct
actgctcggatttggaggtt

SEQ ID NO: 65 - DNA, 21 bp, Artificial Sequence
  Synthetic construct
aaggcagtgaacctcacaaag

SEQ ID NO: 66 - DNA, 19 bp, Artificial Sequence
  Synthetic construct
gccatttggaagagcaggt

SEQ ID NO: 67 - DNA, 21 bp, Artificial Sequence
  Synthetic construct
ccgtcactcaaagcatcaata

SEQ ID NO: 68 - DNA, 19 bp, Artificial Sequence
  Synthetic construct
caggtgctggttcatttgg

SEQ ID NO: 69 - DNA, 23 bp, Artificial Sequence
  Synthetic construct
cgttagttttattgaacgaatgc

SEQ ID NO: 70 - DNA, 21 bp, Artificial Sequence
  Synthetic construct
tctggatattcggtttgaagc

SEQ ID NO: 71 - DNA, 21 bp, Artificial Sequence
  Synthetic construct
atgcgcactttccagttctta

SEQ ID NO: 72 - DNA, 21 bp, Artificial Sequence
  Synthetic construct
caaatgttggttgtctgatgc

SEQ ID NO: 73 - DNA, 21 bp, Artificial Sequence
  Synthetic construct
ggctcaagcaatgtctcgtat

SEQ ID NO: 74 - DNA, 21 bp, Artificial Sequence
  Synthetic construct
tgatgaatttgcgtaaaggtg

SEQ ID NO: 75 - DNA, 21 bp, Artificial Sequence
  Synthetic construct
ggaaagacttggttcttggct

SEQ ID NO: 76 - DNA, 22 bp, Artificial Sequence
  Synthetic construct
cgtaaaggtggcaaattttgaa

SEQ ID NO: 77 - DNA, 20 bp, Artificial Sequence
  Synthetic construct
cattggaacattgggcaaac

SEQ ID NO: 78 - DNA, 21 bp, Artificial Sequence
  Synthetic construct
gagttgttgaagggagcagaa

SEQ ID NO: 79 - DNA, 21 bp, Artificial Sequence
  Synthetic construct
ttgggcaaacgagcttatatc

SEQ ID NO: 80 - DNA, 20 bp, Artificial Sequence
  Synthetic construct
gagcagaaagccaggagaag

SEQ ID NO: 81 - DNA, 21 bp, Artificial Sequence
  Synthetic construct
caaagccaggattcactgaga

SEQ ID NO: 82 - DNA, 21 bp, Artificial Sequence
  Synthetic construct
gaaactcctccttgagccaaa

SEQ ID NO: 83 - DNA, 22 bp, Artificial Sequence
  Synthetic construct
ttcactgagaaactttggatcg

SEQ ID NO: 84 - DNA, 22 bp, Artificial Sequence
  Synthetic construct
cgacttgttgaacttgtgttgg

SEQ ID NO: 85 - DNA, 19 bp, Artificial Sequence
  Synthetic construct
cacttccggatttgcaatg

SEQ ID NO: 86 - DNA, 20 bp, Artificial Sequence
  Synthetic construct
cgcttcgatagggggtaata

SEQ ID NO: 87 - DNA, 19 bp, Artificial Sequence
  Synthetic construct
gtcctccagcactccattg

SEQ ID NO: 88 - DNA, 22 bp, Artificial Sequence
  Synthetic construct
tgcaaatgcattctcaatacaa

SEQ ID NO: 89 - DNA, 21 bp, Artificial Sequence
  Synthetic construct
cctcatttcaatagctgtcgc

SEQ ID NO: 90 - DNA, 21 bp, Artificial Sequence
  Synthetic construct
tgaatagttccgttggcaagt

SEQ ID NO: 91 - DNA, 19 bp, Artificial Sequence
  Synthetic construct
gtcgccatggcagttctac

SEQ ID NO: 92 - DNA, 21 bp, Artificial Sequence
  Synthetic construct
caagtggtacaaacgcatgaa

SEQ ID NO: 93 - DNA, 22 bp, Artificial Sequence
  Synthetic construct
tccaggaagttcaaatcatcaa

SEQ ID NO: 94 - DNA, 21 bp, Artificial Sequence
  Synthetic construct
tgtcttctgattggtggttgc

SEQ ID NO: 95 - DNA, 22 bp, Artificial Sequence
  Synthetic construct
tcaaatcatcaaggatgaacca

SEQ ID NO: 96 - DNA, 20 bp, Artificial Sequence
  Synthetic construct
ttgccattgggaatttgagt

SEQ ID NO: 97 - DNA, 21 bp, Artificial Sequence
  Synthetic construct
cggaagctcacacaagaatcc

SEQ ID NO: 98 - DNA, 18 bp, Artificial Sequence
  Synthetic construct
aaaacggcggttgtttcg

SEQ ID NO: 99 - DNA, 19 bp, Artificial Sequence
  Synthetic construct
cacaagaatccgctactcg

SEQ ID NO: 100 - DNA, 19 bp, Artificial Sequence
  Synthetic construct
ttcggaagaccagttaggg

SEQ ID NO: 101 - DNA, 19 bp, Artificial Sequence
  Synthetic construct
tcgtcggaatccttcacct

SEQ ID NO: 102 - DNA, 19 bp, Artificial Sequence
  Synthetic construct
ctcaagcttgtgagccagg

SEQ ID NO: 103 - DNA, 22 bp, Artificial Sequence
  Synthetic construct
ccgattataaaatgccacttcc

SEQ ID NO: 104 - DNA, 21 bp, Artificial Sequence
  Synthetic construct
gagccaggtagagaattgcgt

SEQ ID NO: 105 - DNA, 22 bp, Artificial Sequence
  Synthetic construct
tcccgaaagatcagaatagagg

SEQ ID NO: 106 - DNA, 21 bp, Artificial Sequence
  Synthetic construct
ggtgcacaccgtatttccata

SEQ ID NO: 107 - DNA, 23 bp, Artificial Sequence
  Synthetic construct
cagaatagaggatcgtttcatca

SEQ ID NO: 108 - DNA, 23 bp, Artificial Sequence
  Synthetic construct
ccatatggatcgtagtaggcaga

SEQ ID NO: 109 - DNA, 21 bp, Artificial Sequence
  Synthetic construct
cggaattctcagaagcccata

SEQ ID NO: 110 - DNA, 21 bp, Artificial Sequence
  Synthetic construct
gtgtccagtgaggcaagaaat

SEQ ID NO: 111 - DNA, 21 bp, Artificial Sequence
  Synthetic construct
aagcccatatccttggcttat

SEQ ID NO: 112 - DNA, 21 bp, Artificial Sequence
  Synthetic construct
tcataaggcagtaattgtccg

SEQ ID NO: 113 - DNA, 23 bp, Artificial Sequence
  Synthetic construct
cttgacttttcatatattcccga

SEQ ID NO: 114 - DNA, 22 bp, Artificial Sequence
  Synthetic construct
aaggcgttgtgataacatcagt

SEQ ID NO: 115 - DNA, 21 bp, Artificial Sequence
  Synthetic construct
aacgaattcatctgtggcatc

SEQ ID NO: 116 - DNA, 21 bp, Artificial Sequence
  Synthetic construct
aatggccatccaaatgtgata

SEQ ID NO: 117 - DNA, 21 bp, Artificial Sequence
  Synthetic construct
caaatcaaattttcagcgcac

SEQ ID NO: 118 - DNA, 21 bp, Artificial Sequence
  Synthetic construct
accaggagttttcgtctcgtt

SEQ ID NO: 119 - DNA, 18 bp, Artificial Sequence
  Synthetic construct
gcacccaagaggggacat

SEQ ID NO: 120 - DNA, 21 bp, Artificial Sequence
  Synthetic construct
atgaagggagctttttgtcgt

SEQ ID NO: 121 - DNA, 21 bp, Artificial Sequence
  Synthetic construct
gttacagcacgcgtcattttt

SEQ ID NO: 122 - DNA, 20 bp, Artificial Sequence
  Synthetic construct
gagctcagtgcattctgtcg

SEQ ID NO: 123 - DNA, 21 bp, Artificial Sequence
  Synthetic construct
cgtcatttttagggcttgatg

SEQ ID NO: 124 - DNA, 21 bp, Artificial Sequence
  Synthetic construct
ggccatccacatagtgtcatt

SEQ ID NO: 125 - DNA, 21 bp, Artificial Sequence
  Synthetic construct
ggccatccacatagtgtcatt

SEQ ID NO: 126 - DNA, 21 bp, Artificial Sequence
  Synthetic construct
atcgtccactgcgatattcat

SEQ ID NO: 127 - DNA, 21 bp, Artificial Sequence
  Synthetic construct
atcgtccactgcgatattcat

SEQ ID NO: 128 - DNA, 23 bp, Artificial Sequence
  Synthetic construct
tgtcctggtactcataatcgaaa

SEQ ID NO: 129 - DNA, 20 bp, Artificial Sequence
  Synthetic construct
gccgctatcggataatgatg

SEQ ID NO: 130 - DNA, 22 bp, Artificial Sequence
  Synthetic construct
gaagactatcagactgcccacc

SEQ ID NO: 131 - DNA, 23 bp, Artificial Sequence
  Synthetic construct
tgtgattatgctttactcgctga

SEQ ID NO: 132 - DNA, 21 bp, Artificial Sequence
  Synthetic construct
ccaccgggaagtacattgtta

SEQ ID NO: 133 - DNA, 19 bp, Artificial Sequence
  Synthetic construct
cgcgcatatgtctttttcc

SEQ ID NO: 134 - DNA, 18 bp, Artificial Sequence
  Synthetic construct
gcgcgcgtcattatttct

SEQ ID NO: 135 - DNA, 22 bp, Artificial Sequence
  Synthetic construct
tgtctttttccagtggtagtgg

SEQ ID NO: 136 - DNA, 21 bp, Artificial Sequence
  Synthetic construct
attatttctcacggcttcgtc

SEQ ID NO: 137 - DNA, 22 bp, Artificial Sequence
  Synthetic construct
ttcgttcagcctatgaactttg

SEQ ID NO: 138 - DNA, 23 bp, Artificial Sequence
  Synthetic construct
cctccttctctcatacaatcgaa

SEQ ID NO: 139 - DNA, 21 bp, Artificial Sequence
  Synthetic construct
ctttgtttacgagcttccggt

SEQ ID NO: 140 - DNA, 21 bp, Artificial Sequence
  Synthetic construct
caatcgaaatcagcattgtct

SEQ ID NO: 141 - DNA, 21 bp, Artificial Sequence
  Synthetic construct
gacaaaggttacagcgacagc

SEQ ID NO: 142 - DNA, 21 bp, Artificial Sequence
  Synthetic construct
tgtctacgttgagcaagatcc

SEQ ID NO: 143 - DNA, 19 bp, Artificial Sequence
  Synthetic construct
cagcgacagcaaagtggtc

SEQ ID NO: 144 - DNA, 21 bp, Artificial Sequence
  Synthetic construct
gcaagatccgtcaatgtgttt

SEQ ID NO: 145 - DNA, 21 bp, Artificial Sequence
  Synthetic construct
ccaatgtagtcatgacaactg

SEQ ID NO: 146 - DNA, 21 bp, Artificial Sequence
  Synthetic construct
gaccactgacgccaaatctgg

SEQ ID NO: 147 - DNA, 22 bp, Artificial Sequence
  Synthetic construct
cacgtggctgcactaattttgc

SEQ ID NO: 148 - DNA, 22 bp, Artificial Sequence
  Synthetic construct
gactcggacggttgcattgagc

SEQ ID NO: 149 - DNA, 22 bp, Artificial Sequence
  Synthetic construct
gcttatcatcataggtttctgc

SEQ ID NO: 150 - DNA, 21 bp, Artificial Sequence
  Synthetic construct
ctgcttgtccgtcataatacc

SEQ ID NO: 151 - DNA, 22 bp, Artificial Sequence
  Synthetic construct
gactcttccgacgattcagatg

SEQ ID NO: 152 - DNA, 22 bp, Artificial Sequence
  Synthetic construct
gattcagatgactgagcaaatc

SEQ ID NO: 153 - DNA, 21 bp, Artificial Sequence
  Synthetic construct
ggatattgtattgaacgttgg

SEQ ID NO: 154 - DNA, 21 bp, Artificial Sequence
  Synthetic construct
ggtggtatgccaactcgaacg

SEQ ID NO: 155 - DNA, 20 bp, Artificial Sequence
  Synthetic construct
acgttggacgtggacatgcg

SEQ ID NO: 156 - DNA, 22 bp, Artificial Sequence
  Synthetic construct
tatgccaactcgaacgcgatgc

SEQ ID NO: 157 - DNA, 22 bp, Artificial Sequence
  Synthetic construct
catttcgttggcgatgctactc

SEQ ID NO: 158 - DNA, 22 bp, Artificial Sequence
  Synthetic construct
ctctttacattgaaaatgaaca

SEQ ID NO: 159 - DNA, 22 bp, Artificial Sequence
  Synthetic construct
gtattatcgaaagtatcagaag

SEQ ID NO: 160 - DNA, 21 bp, Artificial Sequence
  Synthetic construct
tccttcatcatttttatatgt

SEQ ID NO: 161 - DNA, 22 bp, Artificial Sequence
  Synthetic construct
agtatcagaagttcaaatttgg

SEQ ID NO: 162 - DNA, 21 bp, Artificial Sequence
  Synthetic construct
tgtaaatttgataaggtatag

SEQ ID NO: 163 - DNA, 22 bp, Artificial Sequence
  Synthetic construct
gacctggctgaggcacacgatg

SEQ ID NO: 164 - DNA, 21 bp, Artificial Sequence
  Synthetic construct
cagcaacagcaaccaccttcc

SEQ ID NO: 165 - DNA, 20 bp, Artificial Sequence
  Synthetic construct
gagcttgttccggattcgtg

SEQ ID NO: 166 - DNA, 21 bp, Artificial Sequence
  Synthetic construct
ccttccgagcaggagcacaac